(19) United States
(12) Patent Application Publication
(10) Pub. No.: US 2002/0128660 A1
(43) Pub. Date: Sep. 12, 2002

(54) Apparatus particularly for operative placement of surgical instruments in cavities under controlled pressure

(75) Inventor: Gianfranco Bidoia, Padova (IT)

Correspondence Address: MODIANO & ASSOCIATI, Via Meravigli, 16, MILANO, 20123, IT

(73) Assignee: BIDOIA s.a.s. DI GIANFRANCO BIDOIA E C.

(21) Appl. No.: 10/043,253

(22) Filed: Jan. 14, 2002

(30) Foreign Application Priority Data
Jan. 26, 2001 (IT) PD2001A000017

(51) Int. Cl. A61F 11/00; A61M 5/178

(52) U.S. Cl. 606/108; 604/167.01

(57) ABSTRACT

A apparatus particularly for the operative placement of surgical instruments in cavities under controlled pressure in the surgical field or in the medical field in general, of the type that comprises a hollow body suitable to provide an access pathway to a cavity under controlled pressure, further comprising at least two sealing gaskets made of elastically deformable material and contained in the hollow body; the gaskets are coupled at their perimetric portion, are flat, and are arranged in succession coaxially; each gasket is provided with at least three radial cuts. The radial cuts of a same sealing gasket are staggered with respect to those of the adjacent sealing gasket.

### BACKGROUND OF THE INVENTION

The present invention relates to an apparatus particularly for the operative placement of surgical instruments in cavities under controlled pressure, in the surgical field or in the medical field in general.

In the surgical field there is often the need to operate inside body cavities, connecting them to the outside of the body and maintaining therein a certain pressure value, which is sometimes generated by gas introduced artificially.

Laparoscopy, also known as minimally-invasive surgery, is part of this particular field, and is performed by using particular surgical instruments which differ in terms of shape, length and diameter.

In order to be inserted in the human body, these instruments require an insertion pathway, which necessarily has to be produced artificially.

This pathway is obtained by means of apparatuses, known commercially also by the name “trocar”, having various dimensions and shapes, which allow to directly connect the inside of the body to the outside while allowing the passage of such surgical instruments for laparoscopy.

The apparatuses can be constituted by a hollow head, to be arranged outside the body of the patient, such head being coaxially provided with a longitudinally elongated duct with an exit opening to be arranged at a cavity to be inspected of the body of the patient.

Laparoscopy instruments are inserted through an opening formed in the hollow head and are then passed through the straight duct.

In most cases, the cavities of the human body, especially abdominal and pelvic ones, are merely virtual and it is therefore necessary to obtain them with artificial systems, the commonest being the insufflation of a gas, advantageously carbon dioxide, at a controlled pressure.

It is therefore essential that these apparatuses for inserting laparoscopy instruments be able to maintain this gaseous pressure without particular gas leakages, which would force to introduce further gas, with consequent excessive absorption of gas at the organic level.

One type of “trocar” that is currently in use is constituted by a hollow head to be arranged outside the body of the patient and below which there is a duct for the passage of the surgical instrument for laparoscopy, which is meant to connect the outside to the inside of the abdominal and/or pelvic cavity.

At the hollow head there are means that allow to maintain an adequate barometric pressure inside the cavity.

An inlet for the gas under controlled pressure is arranged on the head below said means.

The means for maintaining the pressure inside the cavity can be mechanical, of the laterally-hinged or guillotine type, optionally actuated from outside; once the laparoscopy instrument has passed, for extraction or insertion, such means close the internal channel of the trocar, preventing the escape of the gas from the inside.

The mechanical means allow the surgical instrument to pass together with whatever it is carrying, up to the maximum diameter of said trocar.

However, there are some limitations, such as the complexity of the mechanical part, an increase in the overall dimensions of the trocar, an increase in weight, especially of the head, the part that remains external and extended, considerable difficulties for cleaning and sterilization, and an increase in production costs.

Disposable trocars, instead, especially because of the need to minimize cost and weight, use gaskets made of elastically deformable plastics arranged at the hollow head.

The gaskets can have various shapes, for example a frustum-like shape, in which the narrower portion directed toward the axial duct of the “trocar”; this narrower portion can assume various shapes, i.e., cylindrical with a series of iris-like slits, blade-like with a single cut along said blade, conical with a cut that can be double or radial, extending from the tip and continuing toward the base, for the extent deemed necessary.

These solutions allow to use materials having low thicknesses, since indeed because of their particular shape they cannot be deformed to the point of retracting toward the hollow head of the trocar, i.e., from where the laparoscopy instruments are inserted.

The gaskets used can also be flat, with iris-like cuts; this solution must use considerable thicknesses (3 mm or more), since the gasket, by being flat, must contrast the pressure inside the cavity only by means of the elastic force provided by its own thickness.

Despite their manufacturing simplicity, plastic gaskets are not free from drawbacks.

If they are frustum-shaped, they are in fact coupled so as to always adhere very tightly to the stem of the laparoscopy instrument, tending to tear when the instrument is conveying outward material taken from the cavity.

If instead the gasket is flat, its great thickness tends to keep the sections highly adherent to the stem of the surgical instrument with the points downward; in this case, too, they tend to tear when the surgical instrument is conveying material outward.

### SUMMARY OF THE INVENTION

The aim of the present invention is to solve or substantially reduce the problems of known types of apparatus for the operative placement of surgical and/or medical instruments, of the type suitable to maintain a controlled gas pressure inside the operating cavity.

Within this aim, an object of the present invention is to provide an apparatus that requires no mechanism or manual intervention of any kind in order to maintain a controlled pressure of the gas used.

Another object is to provide an apparatus whose dimensions are not larger than those of known types of apparatus.

A further object is to provide an apparatus both of the throwaway type and of the reusable type.

A still further object is to provide an apparatus that is reliable, in terms of maintaining gas pressure, and is suitable to allow the passage of surgical instruments for laparoscopy, both for insertion and for extraction, even if they are partially open.

Another object is to provide an apparatus that has a simple structure and a low cost.

This aim and these and other objects that will become better apparent hereinafter are achieved by an apparatus particularly for the operative placement of surgical instruments in cavities under controlled pressure in the surgical field or in the medical field in general, of the type that comprises a hollow body suitable to provide an access pathway to a cavity under controlled pressure, characterized in that it comprises at least two sealing gaskets made of elastically deformable material and contained in said hollow body, said gaskets being flat and coupled at their perimetric portion, said gaskets being arranged in succession coaxially, each gasket being provided with at least three radial cuts, said radial cuts of a same sealing gasket being staggered with respect to those of an adjacent sealing gasket.

### BRIEF DESCRIPTION OF THE DRAWINGS

Further characteristics and advantages of the invention will become better apparent from the description of a preferred but not exclusive embodiment thereof, illustrated only by way of non-limitative example in the accompanying drawings, wherein:

FIG. 1 is a perspective view of an apparatus according to the invention with which an insertion stylet is associated;

FIG. 2 is a transverse sectional view of the apparatus of FIG. 1;

FIG. 3 is an exploded view of the apparatus according to the invention;

FIG. 4 is a detailed view of a portion of the exploded view of FIG. 3;

FIGS. 5 and 6 are two different sectional plan views of the apparatus shown in the preceding figures;

FIG. 7 is a view of the lower portion of an apparatus according to the invention, with which a surgical instrument for laparoscopy is associated.

### DESCRIPTION OF THE PREFERRED EMBODIMENTS

With reference to the figures, an apparatus particularly for the operative placement of surgical instruments in cavities under controlled pressure in the surgical field or in the medical field in general is generally designated by the reference numeral 10.

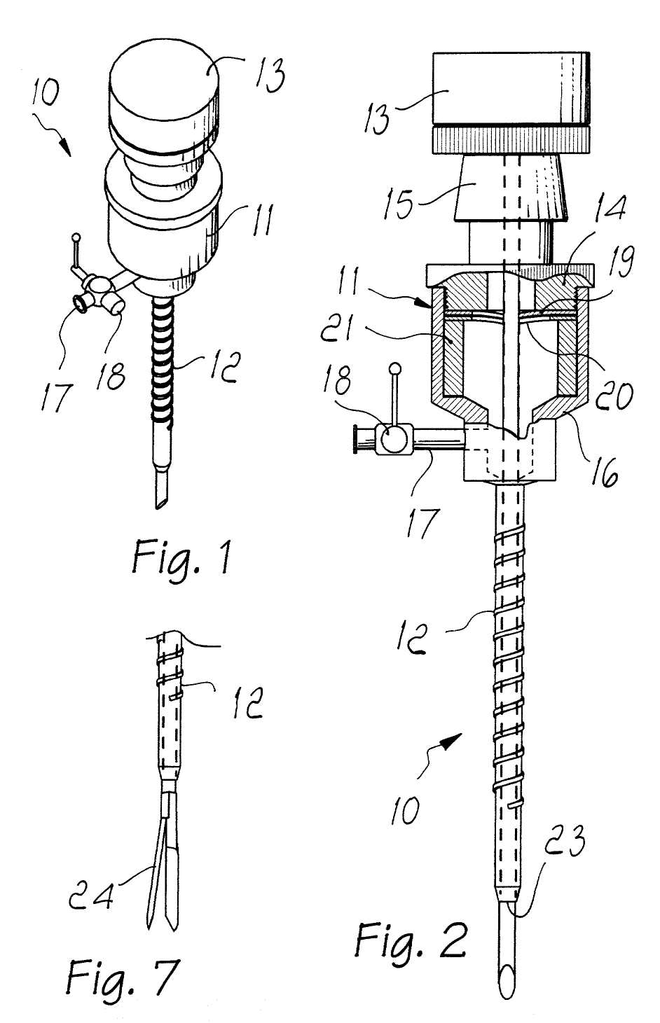

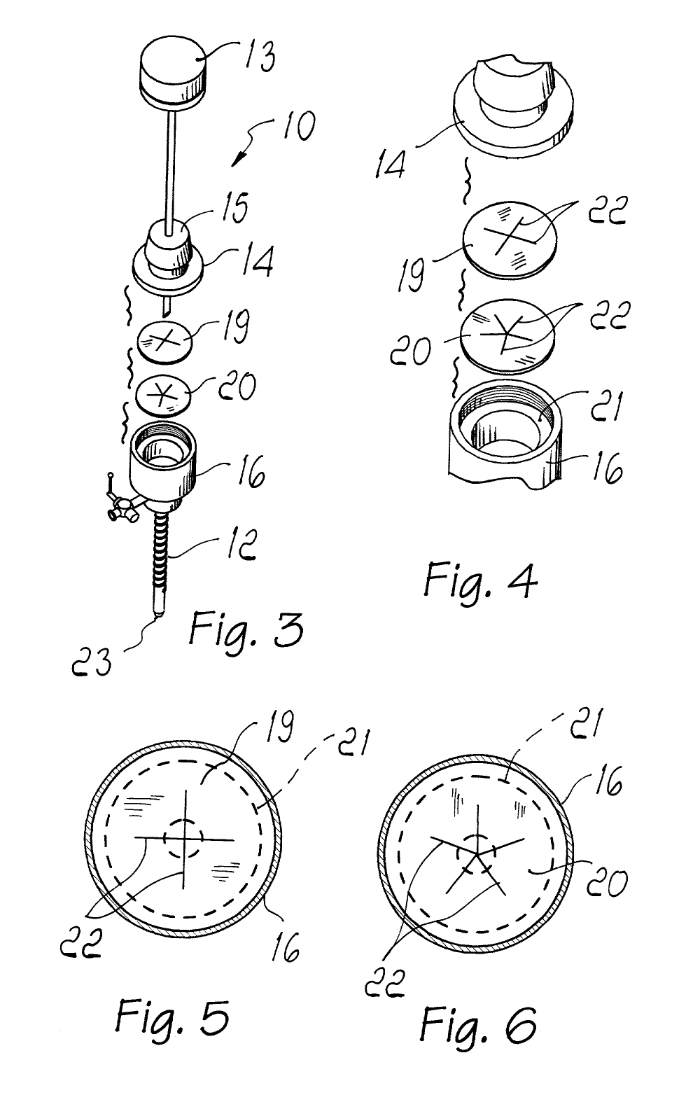

The apparatus 10 comprises a substantially cylindrical hollow head 11, from which an axial duct 12 protrudes downward for the passage, in this case, as shown in FIGS. 1 to 3, of an insertion stylet, generally designated by the reference numeral 13.

The hollow head 11 is constituted by an upper portion 14, provided with a hood-like gasket 15 that is associated, with a screw-and-nut coupling, with a substantially cylindrical hollow lower portion 16.

Below the cylindrical portion 16 there is an inlet 17 for the gas under controlled pressure, provided with a valve 18 for controlling the gas flow.

A first gasket 19 and a second gasket 20, made of elastically deformable material, are arranged inside the cylindrical portion 16.

The first and second gaskets 19 and 20 are flat, with a circular plan shape, and have a thickness of preferably 1 to 2 mm.

The gaskets are arranged so as to be coaxial and superimposed and are coupled with their perimetric portion between the lower base of the upper portion 14 of the head 11 and the upper end surface of a tubular element 21 arranged inside the cylindrical lower portion 16 of the head 11.

The first gasket 19 and the second gasket 20 are provided with radial cuts 22, which extend from the center thereof until they assume a dimension that is at least equal to the inside diameter of the axial duct 12 for the passage of a laparoscopy instrument 24, as shown in FIG. 7.

In particular, in this case, the first gasket 19 is provided with four radial cuts 22 arranged in a cross-like shape, while the underlying second gasket 20 is provided with five equidistant radial slits arranged in a star pattern.

As regards operation, the apparatus 10 is arranged on a patient, leaving outside the head 11 and ensuring that the outlet 23 of the axial duct 12 is arranged at an abdominal or pelvic portion.

Through the inlet 17, with the flow control valve 18 open, gas is introduced in order to obtain a chosen operating cavity.

Then a surgical laparoscopy instrument 24 is inserted through the apparatus 10.

In particular, the apparatus 10 is characterized by an inside diameter of the head 11 and of the axial duct 12 that allows the passage of a laparoscopy instrument 24 even if it is partially open, since it is suitable to convey biological or other material from the inside of the cavity outward or from the outside inward.

Although the first and second gaskets 19 and 20 have very low thicknesses, the escape of the gas is prevented, since the radial cuts 22 of each gasket do not coincide when they overlap, generating in practice a single gasket without cuts or gaps, and with the same sealing capacity as any other kind of gasket of greater thickness.

The gaskets 19 and 20 allow the passage of the surgical instruments and of whatever they are conveying, since the thickness of these gaskets is adequately low.

This allows the central points generated by the radial cuts 22 to flex outward in the direction in which the instrument, for example an insertion stylet 13 or a laparoscopy instrument 24, is passing, and then immediately resume their initial position due to elastic return, without any mechanical or manual intervention.

The fact of having two thin gaskets allows greater adaptability than the solution with a single gasket whose thickness is the sum of the two.

In practice it has been found that the present invention has achieved the intended aim and objects.

An apparatus has in fact been provided which has a simple structure, solves the problem of maintaining the pressure inside the operating cavity, during operating conditions and during the handling of the surgical or medical instruments in general.

The apparatus furthermore allows easy and complete cleaning and sterilization.

The gaskets 19 and 20 can of course have a different plan shape and can be arranged coaxially and spaced, so long as their distance is not greater than the thickness of the largest gasket.

The present invention is susceptible of numerous modifications and variations, all of which are within the scope of the inventive concept.

The technical details may be replaced with other technically equivalent elements.

The materials, so long as they are compatible with the contingent use, as well as the dimensions, may be any according to requirements.

The disclosures in Italian Patent Application No. PD2001A000017 from which this application claims priority are incorporated herein by reference.

What is claimed is:

1. An apparatus particularly for the operative placement of surgical instruments in cavities under controlled pressure in the surgical field or in the medical field in general, of the type that comprises a hollow body suitable to provide an access pathway to a cavity under controlled pressure, further comprising at least two sealing gaskets made of elastically deformable material and contained in said hollow body, said gaskets being flat and coupled at their perimetric portion, said gaskets being arranged in succession coaxially, each gasket being provided with at least three radial cuts, said radial cuts of a same sealing gasket being staggered with respect to those of the adjacent sealing gasket.

2. The apparatus according to claim 1, further comprising a hollow head to be arranged outside a patient and from which an axial duct protrudes downward for the passage of a surgical instrument for laparoscopy, said at least two sealing gaskets being arranged inside said hollow head, said radial cuts extending from the center of the gasket so as to assume a dimension that is at least equal to an inside diameter of a passage duct for a surgical instrument for laparoscopy, the radial cuts of a same sealing gasket being staggered with respect to the cuts of the adjacent sealing gasket.

3. The apparatus according to claim 2, wherein said gaskets are arranged so as to be coaxially adjacent and superimposed and are coupled with their perimetric portion between a lower end face of an upper portion of said hollow head and an abutment surface located inside the lower portion of said head, said upper portion of the hollow head engaging with a reversible coupling on said lower portion.

4. The apparatus according to claim 3, wherein said abutment surface is an upper end face of a tubular element arranged inside the lower portion of said head.

5. The apparatus according to claim 3, wherein said upper portion of the hollow head engages with a screw-and-nut coupling on said lower portion.

6. The apparatus according to claim 1, wherein said gaskets have a substantially circular plan shape.

7. The apparatus according to claim 1, wherein each sealing gasket is provided with a mutually different number of cuts, said number being different by at least one unit.

8. The apparatus according to claim 1, wherein one of said gaskets is provided with four radial cuts arranged in a cross-like pattern and one is provided with five equidistant radial cuts arranged in a star pattern.

9. The apparatus according to claim 1, wherein said gaskets have a thickness between 1 and 2 mm.

10. The apparatus according to claim 9, wherein the maximum total thickness of said gaskets is 4 mm.

11. The apparatus according to claim 1, wherein said gaskets are arranged so as to be axially spaced at a distance that does not exceed the thickness of the largest gasket.
